(19) United States
(12) Patent Application Publication
(10) Pub. No.: US 2021/0322326 A1
(43) Pub. Date: Oct. 21, 2021

(54) HYDROGEL PARTICLE CARRIERS FOR DELIVERY OF THERAPEUTIC/DIAGNOSTIC AGENTS

(71) Applicant: The Board of Trustees of the University of Illinois, Urbana, IL (US)

(72) Inventors: Hyungsoo CHOI, Urbana, IL (US); Kyekyoon KIM, Urbana, IL (US)

(21) Appl. No.: 17/232,336

(22) Filed: Apr. 16, 2021

Related U.S. Application Data
(60) Provisional application No. 63/012,081, filed on Apr. 18, 2020.

(51) Int. Cl. A61K 9/50; A61K 45/06; A61K 47/69

(52) U.S. Cl. CPC A61K 9/5057; A61K 47/6951; A61K 45/06; A61K 9/5089

(57) ABSTRACT

Particles for delivery of therapeutic agents are disclosed. In one embodiment, a particle for delivery of a therapeutic agent includes a hydrogel polymer matrix, a plurality of hydrophobic encapsulation sites within the hydrogel polymer matrix, and a hydrophobic therapeutic agent encapsulated within at least some of the encapsulation sites. The hydrogel polymer matrix may be hydrophilic. The hydrophobic encapsulation sites may comprise cyclodextrin conjugated to the hydrogel polymer matrix. The hydrogel polymer matrix comprises gelatin. The particle may have a diameter of 10 nm to 50 μm.

### CROSS-REFERENCE TO RELATED APPLICATIONS

This application claims the benefit of priority to U.S. Provisional Patent Application No. 63/012,081, filed Apr. 18, 2020, which is hereby incorporated by reference in its entirety.

### BACKGROUND OF INVENTION

It has been challenging to deliver hydrophobic, poorly water-soluble therapeutic or diagnostic agents to water-based or aqueous environments. The approaches to addressing the problems of low aqueous solubility have included the use of salt forms, cocrystals, organic cosolvents, surfactants, complexation agents, emulsions, liposomes, and hydrophobic polymer particles for drug formulations.

Liposomes have been extensively studied as drug carriers for the delivery of hydrophobic drugs. Liposomes are vesicles composed of single or multiple concentric lipid bilayers encapsulating an aqueous compartment. However, liposomes still lack a significant medical impact due to their limited formulation stability, such as aggregation, sedimentation, fusion, phospholipid hydrolysis and/or oxidation, rapid drug release and low drug encapsulation efficiency (EE) in addition to their destabilization by blood lipoproteins, rapid clearance from blood circulation and uptake by reticuloendothelial system. To improve the stability and circulation time, modified liposomes, e.g., pegylated liposomes and cationic liposomes, have been introduced. In addition, to circumvent the low EE and rapid release of hydrophobic drugs, drug-β-cyclodextrin (β-CD) inclusion complexes have been utilized by entrapping them in the aqueous phase of liposomes. β-CDs are water-soluble cyclic oligosaccharides with a hydrophobic cavity that allows to form inclusion complex with hydrophobic molecules. The EE of liposomes may be improved by this, however, the issue of fast drug release still remains.

Another conventional method for the delivery of hydrophobic drugs is gelatin particles loaded with β-CD inclusion complexes of hydrophobic drugs. The hydrophilic nature of gelatin, thwarting the interactions of the particle with blood lipoproteins, results in its long blood-circulation time. In addition, the surface charge of gelatin is controlled by processing conditions, i.e., negatively or positively. This induces electrostatic repulsion between the particles, preventing their aggregation and flocculation. The gelatin particles loaded with drug-β-CD inclusion complexes exhibited slower drug release than each component, CD or gelatin, alone. The rate of drug release from these particles is influenced by the type of CD and gelatin, likely due to the interactions between them. However, the drug release is still fast even in the absence of enzymatic degradation of gelatin.

Accordingly, it can be seen that improved systems and methods for delivery of hydrophobic drugs are needed.

### SUMMARY OF THE INVENTION

Provided herein are improved systems and methods for delivery of hydrophobic drugs. In one embodiment, a particle comprises cyclodextrin conjugated to a gelatin matrix (also referred to herein as “CD-conjugated gelatin”). The advantages of the present invention include the following: the internal cavity of CD is hydrophobic while their outer surface is hydrophilic, allowing to entrap hydrophobic drugs to form inclusion complexes. The CD molecules covalently conjugated to gelatin remain intact and are released as gelatin degrades. The degradation rate is controlled by the particle size and the extent of gelatin crosslinking. As a result, drug release from these CD-conjugated gelatin particles is sustained significantly in comparison to the gelatin particles loaded with β-CD inclusion complexes.

In one aspect, the particle is a hydrophilic polymer particle with hydrophobic pockets in the polymer matrix which can carry chemical molecules. The nano/micro carriers allow delivery of therapeutic and/or diagnostic agents of low aqueous solubility. The hydrophobic molecules are confined inside the hydrophobic pockets and released. The release of molecules may be facilitated as the polymer matrix degrades.

The disclosed systems and methods offer improved solutions to the problems of delivering poorly water-soluble molecules, employing hydrogel particles that have hydrophobic pockets in the polymer matrix. The disclosed systems and methods allow for encapsulation of hydrophobic agents or co-encapsulation of hydrophobic agents with hydrophilic agents and delivery via aqueous media. The agents encapsulated in the particles are released by diffusion that may be facilitated by polymer degradation, allowing sustained delivery.

In one embodiment, the release of hydrophobic agents may be retarded due to the hydrophobic pockets of cyclodextrin being conjugated to the hydrogel polymer. Polymer crosslinking may further play a role in the management of hydrophobic agent release.

In one embodiment, a particle for delivery of a therapeutic agent comprises a hydrogel polymer matrix, a plurality of hydrophobic encapsulation sites within the hydrogel polymer matrix, and a hydrophobic therapeutic agent encapsulated within at least some of the encapsulation sites. The hydrogel polymer matrix may be hydrophilic.

The hydrophobic encapsulation sites may comprise cyclodextrin conjugated to the hydrogel polymer matrix.

In one embodiment, the cyclodextrin of the hydrophobic encapsulation sites is covalently conjugated to the hydrogel polymer matrix

In one embodiment, the hydrogel polymer matrix comprises gelatin. In one embodiment, the particle is a nanoparticle. In one embodiment, the particle has a diameter of 10 nm to 50 μm. In one embodiment, the particle has a diameter of 50 nm to 1000 nm. In one embodiment, the particle has a diameter of 100 nm to 500 nm.

In one embodiment, the particle is microparticle. For example, in one embodiment, the particle has a diameter of 1 μm to 1000 μm. In another embodiment, the particle has a diameter of 50 μm to 700 μm. In another embodiment, the particle has a diameter of 100 μm to 500 μm.

In one embodiment, the hydrogel polymer matrix comprises gelatin, and the cyclodextrin comprises cyclodextrin conjugated to the gelatin of the hydrogel polymer matrix. In one embodiment, the hydrogel polymer matrix is crosslinked. In one embodiment, the hydrogel polymer matrix is crosslinked via glutaraldehyde. In one embodiment, the cyclodextrin is beta-cyclodextrin

In one embodiment, a method of controlling delivery of a hydrophobic therapeutic agent comprises, providing a particle, the particle comprising a hydrophilic polymer matrix having a plurality of hydrophobic encapsulation sites therein, the hydrophobic encapsulation sites comprising cyclodextrin conjugated to the hydrogel polymer matrix, and a hydrophobic therapeutic agent encapsulated within at least some of the encapsulation sites. The method may comprise delivering the particle to a target site, releasing the hydrophobic therapeutic agent, wherein the releasing comprises degrading the hydrophilic polymer matrix in a liquid environment, dissociating the cyclodextrin pockets from the hydrogel matrix of the particle.

In one embodiment, the method comprises controlling a release rate of therapeutic agent from the hydrophobic encapsulation sites by adjusting at least one of: a diameter of the particle, and/or extent of crosslinking of the hydrophilic polymer matrix of the particle. In one embodiment, the releasing step comprises diffusion of the hydrophobic therapeutic from the hydrophilic polymer matrix into the liquid environment.

In one embodiment, the hydrogel polymer matrix comprises gelatin. In one embodiment, the particle has a preselected diameter of 50 nm to 1000 nm. In one embodiment, the particle has a preselected diameter of 100 nm to 500 nm.

In one embodiment, a method of making particles for delivery of a hydrophobic therapeutic agent comprises activating a cyclodextrin feedstock to produce an activated cyclodextrin product, conjugating the activated cyclodextrin product with a hydrogel polymer to produce a conjugated hydrogel polymer, and precipitating particles from the conjugated hydrogel polymer, the particles comprising a hydrogel polymer matrix having hydrophobic encapsulation sites therein.

In one embodiment, the step of activating a cyclodextrin feedstock comprises contacting the cyclodextrin with an imidazole. In one embodiment, the method comprises crosslinking the hydrogel polymer matrix of the particles. In one embodiment, the crosslinking comprises contacting the particles with glutaraldehyde. In one embodiment, the step of precipitating particles from the conjugated hydrogel polymer comprises contacting the conjugated hydrogel polymer mixture with acetone.

In one embodiment, the step of conjugating the activated cyclodextrin product comprises contacting the activated cyclodextrin product with the hydrogel polymer at a temperature of from 30 to 60° C. for a duration of 12 to 30 hours. In one embodiment, the hydrogel polymer matrix comprises gelatin. In one embodiment, the precipitating step comprises controlling precipitation conditions to create particles having a predetermined size distribution peak for diameter of from 50 nm to 1000 nm.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1. Scanning electron micrographic images of β-cyclodextrin conjugated gelatin nanoparticles (β-CD-GNPs) of sizes of 25-500 nm in diameter. Scale bar=200 nm.

FIG. 2. Estrogen (E2)-release profiles of β-cyclodextrin-conjugated gelatin nanoparticles vs. β-cyclodextrin-loaded gelatin nanoparticles of sizes of 200 nm (left) and 370 nm (right) in diameter (n=4).

FIGS. 3A-3C. In vitro evaluation of β-cyclodextrin conjugated gelatin nanoparticles (β-CD-GNPs). Scanning electron micrograph of β-cyclodextrin conjugated gelatin nanoparticles (β-CD-GNPs) (FIG. 3A) and after (FIG. 3B) estrogen loading. Release of estrogen from different particle formulations (FIG. 3C) as a percent of total amount released, n=4. WS-E2: water soluble-E2 (inclusion complex of β-CD with E2); WS-E2 GNP: WS-E2 loaded GNPs; β-CD-GNP: E2-loaded β-cyclodextrin conjugated GNPs. Error bars represent standard deviation.

FIGS. 4A-4B. Effect of intranasal estrogen on infarct volume: Comparison of 100 ng E2-loaded β-cyclodextrin conjugated gelatin nanoparticles (E2-GNPs), 100 ng WS-E2-loaded GNPs (WS-E2) in WT mice (FIG. 4A), and representative images (FIG. 4B). Error bars represent standard error of the mean, n=4, *p<0.05.

FIG. 5 shows in vitro bile acid release profiles of two different sizes of β-CD-conjugated GNPs.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

As used herein, “hydrophobic encapsulation sites” means molecular sites within the hydrogel polymer matrix, where in the molecular sites act as pockets of hydrophobic regions within the otherwise hydrophilic hydrogel polymer matrix. Thus, the hydrophobic encapsulation sites facilitate the encapsulation of hydrophobic materials within a hydrophilic hydrogel. In some embodiments, the hydrophobic encapsulation sites comprise cyclodextrin. The cyclodextrin may be conjugated to the hydrogel polymer matrix. Thus, in some embodiments, hydrophobic material may be encapsulated within a macrocyclic ring of the cyclodextrin, and the cyclodextrin may be conjugated to the hydrogel polymer matrix along the exterior of the macrocyclic ring.

As used herein, a “hydrophobic therapeutic agent” means any hydrophobic material used for the purpose of diagnosis and/or treatment.

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

Provided herein are improved systems and methods for delivery of hydrophobic drugs. In one embodiment, a particle comprises cyclodextrin conjugated to a gelatin (also referred to herein as “CD-conjugated gelatin”). The advantages of the present invention include the following: the internal cavity of CD is hydrophobic while their outer surface is hydrophilic, allowing to entrap hydrophobic drugs to form inclusion complexes. The CD molecules covalently conjugated to gelatin remain intact and are released as gelatin degrades. The degradation rate is controlled by the particle size and the extent of gelatin crosslinking. As a result, drug release from these particles is improved significantly in comparison to gelatin particles loaded with β-CD inclusion complexes.

The invention can be further understood by the following non-limiting examples.

### EXAMPLE 1

### In Vitro Release Study

Fabrication of gelatin particles (GNPs) conjugated with β-cyclodextrin (β-CD)

500 mg of 1,1′-carbonyldiimidazole (CDI) and 100 mg of β-CD were placed in two three-necked flasks, respectively, and vacuum dried overnight. 25 mL of anhydrous dimethylformamide was added to each flask under N₂. The β-CD solution was added dropwise into the CDI solution. The resulting solution was stirred for 24 h at 24° C. under N₂, followed by adding 250 mL of tetrahydrofuran and 750 mL of diethyl ether. Thus-produced precipitates, i.e., imidazolated β-CD, were isolated by filtration and analyzed by ¹H NMR spectrometry. 10-50 mg of imidazolated β-CD was added to 10 mL of 1-5 w/v % aqueous solution of type A gelatin (300 g bloom) at 50° C. and stirred for 24 h.

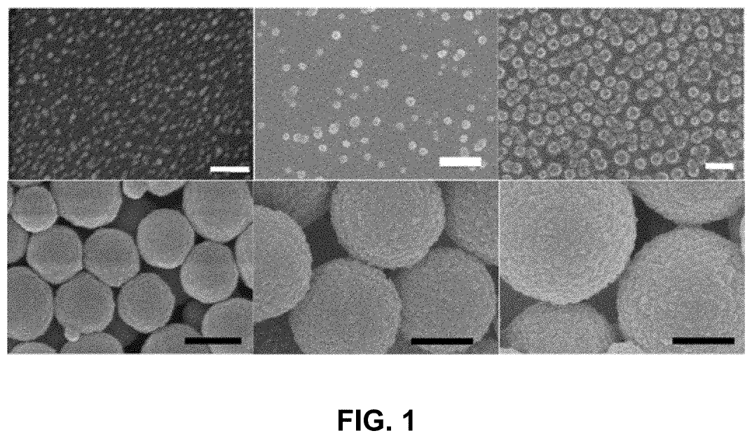

The conjugation of β-CD to gelatin was confirmed by FT-IR and ¹H NMR spectrometry. 50 mL of acetone was added to the solution of β-CD-conjugated gelatin at 50° C., which was stirred until the solution became turbid. To crosslink the resulting β-CD-gelatin particles (β-CD-GNPs), 1.6 mL of 25% glutaraldehyde solution was added to the solution after cooling it down to 25° C., which was stirred for 2 h and washed with 100 mM glycine solution and ethanol via centrifugation. Thus-obtained β-CD-GNPs were lyophilized. FIG. 1 shows scanning electron micrographic images of β-CD-GNPs of different sizes.

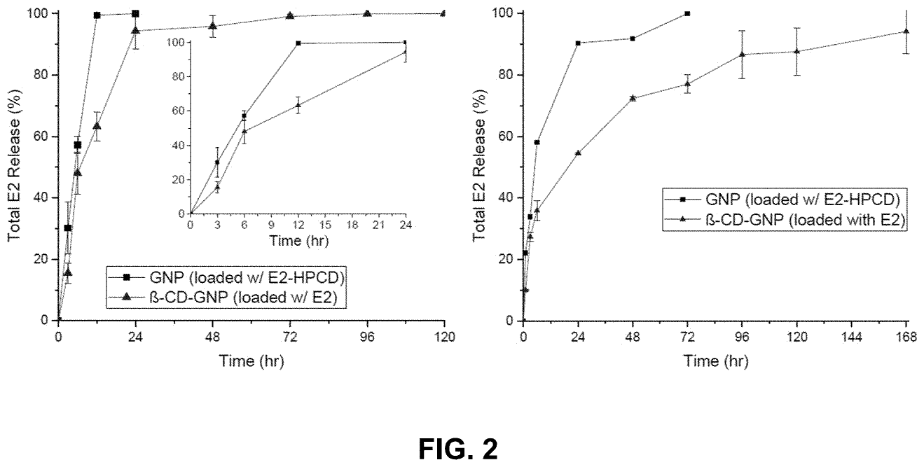

1 mg of β-CD-GNPs were loaded with 15 μg of 17 β-estradiol (E2) in 1 ml of PBS solution to prepare E2-loaded β-CD-GNPs (E2-β-CD-GNPs) solution. The release experiment was performed by incubating the E2-GNP solution in a shaking incubator at 37° C. in the presence of 1,240 ng/ml collagenase. E2 released from the particles was assessed by the absorbance peak at 280 nm using a UV-vis spectrometer as a function of time. The experiment was quadruplicated. FIG. 2 shows cumulative E2 release from β-CD-GNPs of sizes of 200 nm and 370 nm in diameter, demonstrating different E2 release rates. As a control, GNPs with no conjugated β-CD were prepared and loaded with 2-hydroxypropyl-β-cyclodextrin-E2 (E2-HPCD, Sigma-Aldrich) and used for release study. The release rate of E2 decreased with the particle size. As a result, with the 200 nm-sized particles, 100% E2 release was achieved in 12 hours from the GNPs with no β-CD conjugation and 3 days from β-CD-GNPs. With the 370 nm-sized particles, 100% release was achieved in 3 days from the former and >7 days from the latter. E2 release from the GNPs loaded with E2-HPCD was more rapid compared to that from the β-CD-GNPs for both size groups.

### EXAMPLE 2

### In Vivo Study Intranasal Administration

Globally, ischemic stroke is a leading cause of death and adult disability. Previous efforts to repair damaged brain tissue following ischemic events have been hindered by the relative isolation of the central nervous system. A gelatin particle-mediated intranasal drug delivery system was found to be an efficient, non-invasive method for delivering 17β-estradiol (E2) specifically to the brain, enhancing neuroprotection, and limiting systemic side effects. Young adult male C57BL/6J mice subjected to 30 min of middle cerebral artery occlusion (MCAO) were administered intranasal preparations of E2-GNPs, water soluble E2, or saline as control 1 h after reperfusion. Following intranasal administration of 500 ng E2-GNPs, brain E2 content rose by 5.24 fold (p<0.0001) after 30 min and remained elevated by 2.5 fold at 2 h (p<0.05). The 100 ng dose of E2-GNPs reduced mean infarct volume by 54.3% (p<0.05, n=4) in comparison to saline treated controls, demonstrating our intranasal delivery system's efficacy.

Neurologic disorders, such as ischemic stroke, take a significant toll on their victims, as well as burden society as a whole. An estimated 6.2 million people died from stroke in 2015, making it the second leading cause of death globally. In the United States, ischemic stroke is a leading cause of severe adult disability and 5^{th} leading cause of death with an estimated total annual cost of $33.9 billion in 2012. As the population continues to age, the annual cost of stroke is projected to be $184.1 billion by 2030. Interestingly, loss of wages is anticipated to be the single biggest contributor to the total economic burden of stroke, indicating that merely increasing stroke survival is insufficient; preventing long-term disability should be of paramount concern.

Gelatin, a natural polymer of hydrolyzed collagen, is a biocompatible and biodegradable hydrogel that can be formed into mucoadhesive particles. Gelatin particles (GNPs) can be used to encapsulate a variety of therapeutics with the release profiles manipulated by changing particle size and crosslinking. Because E2 is nonpolar and hydrophobic, it does not complex well with GNPs in the aqueous environment required for IN administration. GNPs can instead be loaded with water-soluble 17β-estradiol (WS-E2), which contains 40-55 mg E2 per gram of solid 2-hydroxypropyl-β-cyclodextrin (β-CD). The cyclic oligosaccharides composing β-CD form a hydrophilic exterior shell with a hydrophobic interior cavity that can complex with hydrophobic drugs and improve their solubility in an aqueous particle environment. β-CD can also be conjugated to free amine groups on gelatin before forming GNPs, allowing for direct loading of pure E2.

Fabrication and in vitro analysis of GNPs

β-CD-conjugated GNPs (β-CD-GNPs) with 5 or 10 w/w % β-CD were prepared using a modified desolvation method at 50° C. and pH 2.5, as previously described. GNPs were crosslinked with 0.0625% glutaraldehyde for 2 hours at 25° C. The resulting particles have spherical morphology with average diameter of 372±86 nm. Fourier transform infrared (FTIR) spectra of raw gelatin, 5% β-CD-GNPs, and β-CD were recorded with Nicolet Nexus 670 FTIR spectrophotometer (n=3). Loading efficiency was determined by UV-vis absorption at 280 nm after 2 h with 5% and 10% 13-CD-GNPs achieving 87.43% and 96.41% E2 loading, respectively (n=3). For the in vitro release study, β-CD-GNPs fabricated with gelatin containing 5 or 10 w/w% β-CD were loaded with E2 in a ratio of 1:60. As a comparison, unmodified GNPs were loaded with water soluble E2 (WS-E2) to form WS-E2-GNPs. After a 2 h loading period, samples were immersed in 1 mL PBS with collagenase (1.24 μg/mL) and incubated in a shaking incubator at 37° C. At each time point, supernatant samples were collected and replaced with fresh PBS. The amount of released E2 in PBS was determined by UV-Vis absorption at 280 nm (n=4).

MCAO Surgery

Mice were housed on a 12 h light-dark cycle with ad libitum access to food and water. Animal procedures were performed with strict adherence to all national and institutional guidelines and were approved by the University of Illinois' Institutional Animal Care and Use Committee (protocol #15152). For the stroke model, 3-4 month old male C57BL/6J mice (wild type, WT) from the Jackson Laboratory underwent 30 min of transient MCAO using a modified Koizumi technique, as described previously. Pre- and post-operative blood glucose measurements were made by sampling the lateral tail vein. Post-operatively, mice were housed individually in heated cages with food pellets, water, 15% glucose solution, and food mush freely available and analgesia (0.5 mg/kg carprofen in 1 mL warm saline, subcutaneous) was administered daily. The sham procedure and care are identical except the filament is not inserted.

Intranasal Administration

Minimally anesthetized mice were placed in a supine position in a custom, tube-shaped holder that exposes the nares while inhibiting other movements 1 h after reperfusion. Once restrained, a plastic pipette tip was used to administer 2.5 μL drops to alternating nares. Intranasal treatments in wild type mice were 50, 100, and 500 ng of E2 loaded into 25 μg of β-CD-GNPs (E2-GNPs) in 5 μL PBS, 100 ng of water soluble E2 (WS-E2) in 5 μL PBS, or 5 μL PBS as control with sham mice receiving PBS.

Infarct Volume Measurement

Mice were euthanized by CO₂ asphyxiation and cervical dislocation at 48 h post-MCAO. Six 1 mm coronal sections were collected from each brain using fresh razor blades (Lord Super Stainless) and a pre-chilled mold (acrylic, Zivic Instruments). Slices were immediately stained by immersion in 1% 2,3,5-triphenyl tetrazolium chloride (TTC) at 37° C. for 10 min and fixed in 4% paraformaldehyde. Infarct volumes were automatically determined from images of scanned brain slices.

Statistical Analysis

Two-sample comparisons were performed using the Student's t-test and multiple comparisons by one-way or two-way analysis of variance (ANOVA) followed by

Tukey post hoc testing implemented with R (www.r-project.org). Results are presented as mean±SEM and statistical difference was accepted for β-values <0.05.

In Vitro Analysis of GNPs

FTIR indicated that β-CD was successfully conjugated with gelatin as the 5% β-CD-GNP sample had the same set of peaks as those in β-CD at 1028, 1070, 1150 and 3371 cm⁻¹. These peaks represent CO stretching vibration in primary alcohols, cyclic alcohols, glucosidic bonds, and the symmetric and antisymmetric OH stretching modes of pure β-CD, respectively.

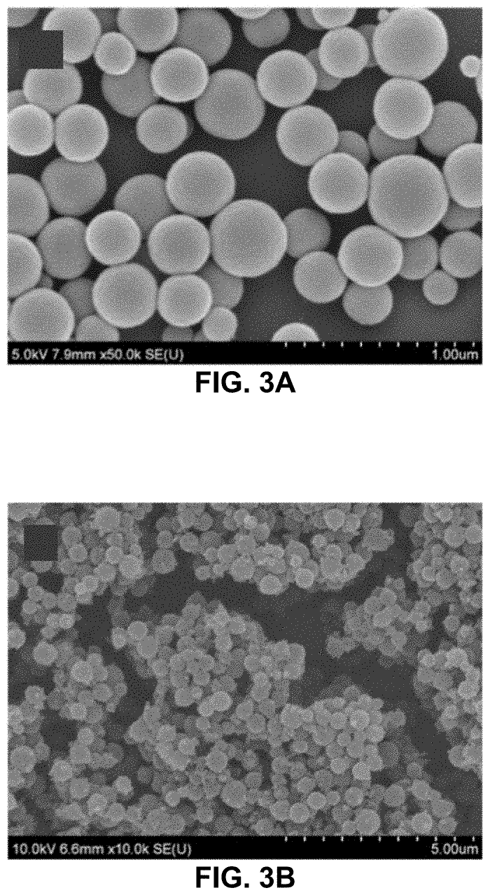

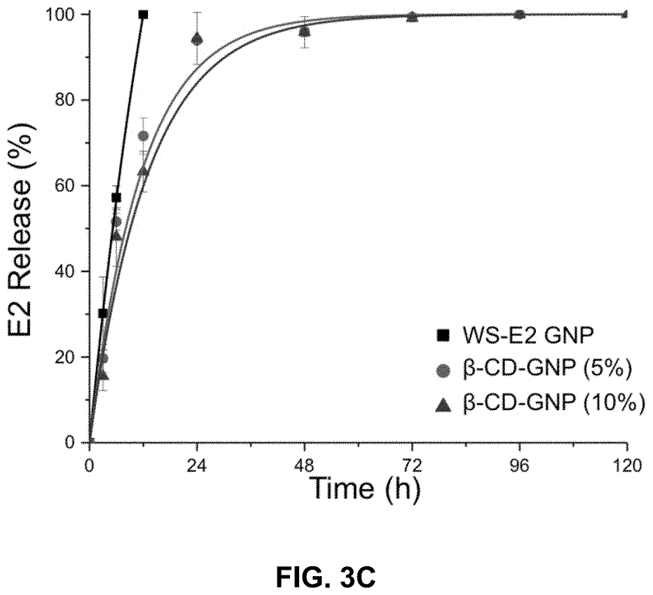

FIG. 3C shows the in vitro release profiles for WS-E2-GNPs, 5% β-CD-GNPs, and 10% β-CD-GNPs. Initial burst release was observed for all samples within the first few hours as un-complexed E2 rapidly diffused out of the GNPs. WS-E2-GNPs had complete E2 within 12 hours; while only about 72% and 63% of E2 were released from the 5% and 10% β-CD-GNPs, respectively. WS-E2 was rapidly released from GNPs under aqueous conditions, whereas incorporating β-CD into the GNP matrix via covalent conjugation extended E2 release to over 48 h with full release not occurring until 120 h in the presence of enzyme. As β-CD content did not impact release rate, 5 w/w% β-CD-GNPs was used for all further studies.

Effect of E2-GNPs on infarct volume

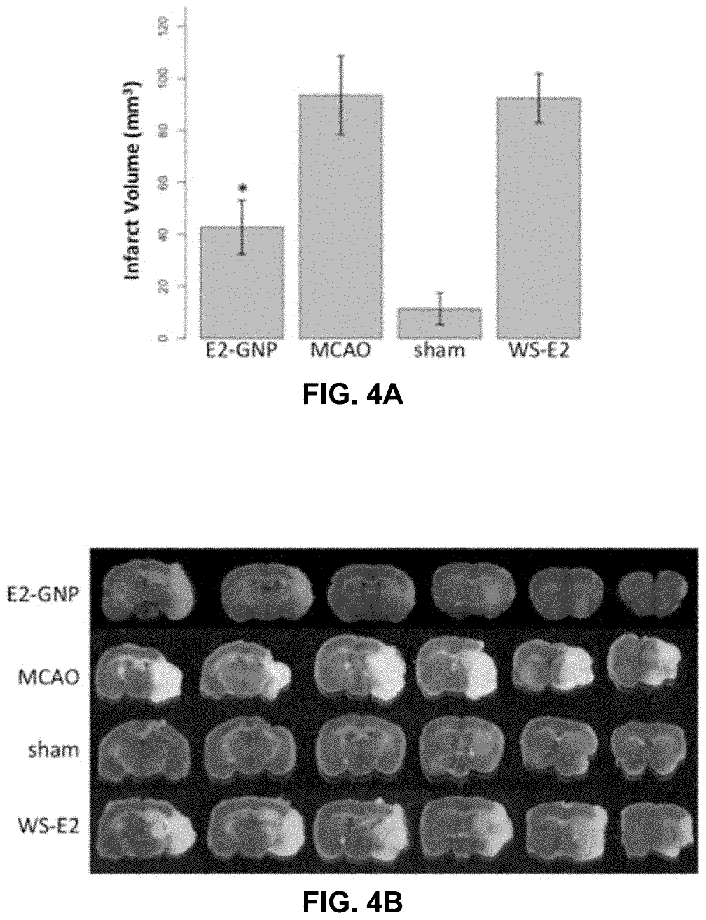

Treatment with 100 ng of E2-GNP resulted in a 54.3% reduction (p<0.05, n=4) in mean infarct volume in WT mice in comparison to MCAO (FIG. 4A-4B).

Effect WS-E2 on Infarct Volume

Treatment with 100 ng of WS-E2 had no effect in WT mice (FIGS. 4A-4B).

### EXAMPLE 3

### In Vitro Release Study: Bile Acid-loaded β-CD-Conjugated Gelatin Nanoparticles

In Vitro Bile Acid release rate over time was studied for two different sizes, 170 and 400 nm diameter, of RCD-conjugated Gelatin Nanoparticles. The bile acid was encapsulated in the βCD-GNPs using the procedure described above.

Deoxycholic acid (DCA), a bile acid (BA), was used for the in vitro release experiment. In other embodiments, other bile acids, such as chenodeoxycholic acid (CDCA), can also be encapsulated in the βCD-GNPs.

For the release study, 2 mg of BA-loaded particles with diameters of 170 and 400 nm were used, showing the release rate controlled by the particle size.

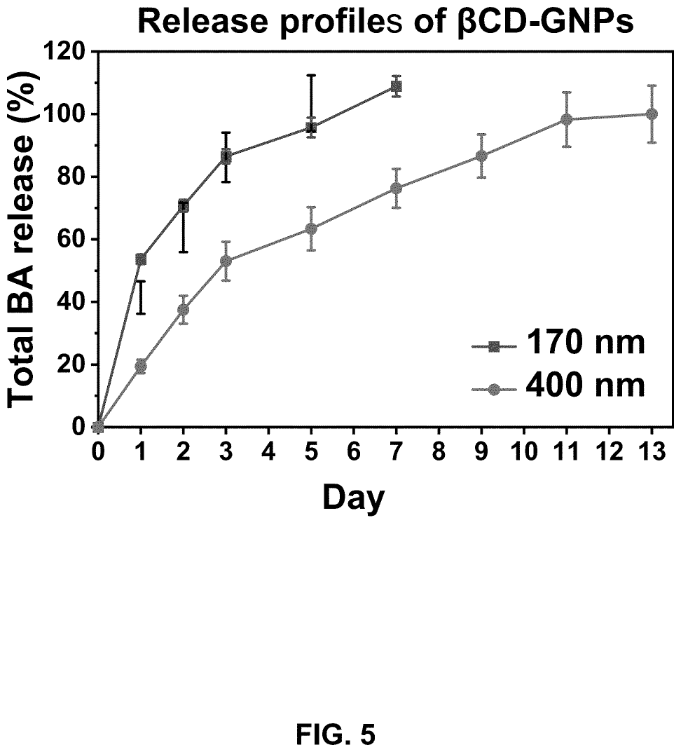

FIG. 5 shows a graph of the release profiles for the two different sizes of nanoparticles.

### REFERENCES

McCormack, B. and Gregoriadis, G. (1994). “Drugs-in-cyclodextrins-in liposomes: a novel concept in drug delivery.” International Journal of Pharmaceutics 112(3): 249-258.

Vandervoort, J. and Ludwig, A. (2004). “Preparation and evaluation of drug-loaded gelatin particles for topical ophthalmic use.” European Journal of Pharmaceutics and Biopharmaceutics 57(2): 251-261.

Boch, R. E., Singh, D. M. R., Karmadi, I. (2014). “Drug delivery system for hydrophobic drugs.” U.S. Pat. No. 8,852,629.

Global Health Estimates 2015: Deaths by Cause, Age, Sex, by Country and by Region, 2000-2015. 2016. Geneva: World Health Organization.

Benjamin E J, Blaha M J, Chiuve S E, Cushman M, Das S R, Deo R, et al. Heart Disease and Stroke Statistics-2017 Update: A Report From the American Heart Association. Circulation 2017. DOI: 10.1161/cir.0000000000000485.

Tabata Y and Ikada Y. Protein release from gelatin matrices. Advanced Drug Delivery Reviews 1998; 31: 287-301. DOI: http://dx.doi.org/10.1016/S0169-409X(97)00125-7.

Joachim E, Kim I-D, Jin Y, Kim K, Lee J-K and Choi H. Gelatin particles enhance the neuroprotective effects of intranasally administered osteopontin in rat ischemic stroke model. Drug Deliv and Transl Res 2014; 4: 395-399. DOI: 10.1007/s13346-014-0208-9.

Cheng F, Choy Y B, Choi H and Kim K. Modeling of small-molecule release from crosslinked hydrogel microspheres: Effect of crosslinking and enzymatic degradation of hydrogel matrix. Int J Pharmaceut 2011; 403: 90-95. DOI: http://dx.doi.org/10.1016/j.ijpharm.2010.10.029.

Choy Y B, Cheng F, Choi H and Kim K. Monodisperse gelatin microspheres as a drug delivery vehicle: Release profile and effect of crosslinking density. Macromol Biosci 2008; 8: 758-765.

Schaschke N, Assfalg-Machleidt I, Machleidt W, Laβleben T, Sommerhoff CP and Moroder L. β-Cyclodextrin/epoxysuccinyl peptide conjugates: a new drug targeting system for tumor cells. Bioorganic & Medicinal Chemistry Letters 2000; 10: 677-680. DOI: https://doi.org/10.1016/S0960-894X(00)00078-0.

Kim I-D, Sawicki E, Lee H-K, Lee E-H, Park HJ, Han P-L, et al. Robust neuroprotective effects of intranasally delivered iNOS siRNA encapsulated in gelatin particles in the postischemic brain. Nanomedicine: Nanotechnology, Biology and Medicine 2016; 12: 1219-1229. DOI: 10.1016/j.nano.2016.01.002.

Morris G P, Wright A L, Tan R P, Gladbach A, Ittner L M and Vissel B. A Comparative Study of Variables Influencing Ischemic Injury in the Longa and Koizumi Methods of Intraluminal Filament Middle Cerebral Artery Occlusion in Mice. PLoS ONE 2016; 11: e0148503. DOI: 10.1371/journal.pone.0148503.

Rodríguez-Tenreiro C, Alvarez-Lorenzo C, Concheiro Â and Torres-Labandeira J J. Characterization of cyclodextrincarbopol interactions by DSC and FTIR. Journal of Thermal Analysis and calorimetry 2004; 77: 403-411. journal article. DOI: 10.1023/B:JTAN.0000038981.30494.f4.

Bonenfant D, Niquette P, Mimeault M, Furtos-Matei A and Hausler R. UV-VIS and FTIR spectroscopic analyses of inclusion complexes of nonylphenol and nonylphenol ethoxylate with β-cyclodextrin. Water Res 2009; 43: 3575-3581. DOI: https://doi.org/10.1016/j.watres.2009.05.010.

Valero M, Pérez-Revuelta B I and Rodriguez L J. Effect of PVP K-25 on the formation of the naproxen:β-ciclodextrin complex. Int J Pharmaceut 2003; 253: 97-110. DOI: https://doi.org/10.1016/S0378-5173(02)00664-6.

Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately.

When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COON) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A particle for delivery of a therapeutic or diagnostic agent, the particle comprising:
a hydrogel polymer matrix;
wherein the hydrogel polymer matrix is hydrophilic;
a plurality of hydrophobic encapsulation sites within the hydrogel polymer matrix;
wherein the hydrophobic encapsulation sites comprise cyclodextrin conjugated to the hydrogel polymer matrix;
a hydrophobic therapeutic agent encapsulated within at least some of the encapsulation sites.

2. The particle of claim 1, wherein the hydrogel polymer matrix comprises gelatin.

3. The particle of claim 1, wherein the particle has a diameter of 10 nm to 50 μm.

4. The particle of claim 1, wherein the particle has a diameter of 50 nm to 1000 nm.

5. The particle of claim 1, wherein the particle has a diameter of 100 nm to 500 nm.

6. The particle of claim 1, wherein the hydrogel polymer matrix comprises gelatin, and wherein the cyclodextrin comprises cyclodextrin conjugated to the gelatin of the hydrogel polymer matrix.

7. The particle of claim 1, wherein the hydrogel polymer matrix is crosslinked.

8. The particle of claim 1, wherein the hydrogel polymer matrix is crosslinked via glutaraldehyde.

9. The particle of claim 1, wherein the cyclodextrin is beta cyclodextrin.

10. The particle of claim 1, wherein the cyclodextrin of the hydrophobic encapsulation sites is covalently conjugated to the hydrogel polymer matrix.

11. A method of controlling delivery of a hydrophobic therapeutic agent, the method comprising:
providing a particle comprising:
a hydrophilic polymer matrix having a plurality of hydrophobic encapsulation sites therein, the hydrophobic encapsulation sites comprising cyclodextrin conjugated to the hydrogel polymer matrix; and
a hydrophobic therapeutic agent encapsulated within at least some of the encapsulation sites;
delivering the particle to a target site;
releasing the hydrophobic therapeutic agent, wherein the releasing comprises degrading the hydrophilic polymer matrix in a liquid environment;
slowing the release of the hydrophobic therapeutic agent conjugated bonds between the cyclodextrin conjugated to the hydrogel polymer matrix of the particle.

12. The method of claim 11, comprising controlling a release rate of therapeutic agent from the hydrophobic encapsulation sites by adjusting at least one of: a diameter of the particle, and/or extent of crosslinking of the hydrophilic polymer matrix of the particle.

13. The method of claim 11, wherein the releasing step comprises diffusion of the hydrophobic therapeutic from the hydrophilic polymer matrix into the liquid environment.

14. The method of claim 11, wherein the hydrogel polymer matrix comprises gelatin.

15. The method of claim 11, wherein the particle has a preselected diameter of 50 nm to 1000 nm.

16. The method of claim 11, wherein the particle has a preselected diameter of 100 nm to 500 nm.

17. A method of making particles for delivery of a hydrophobic therapeutic agent, the method comprising:
activating a cyclodextrin feedstock to produce an activated cyclodextrin product;
conjugating the activated cyclodextrin product with a hydrogel polymer to produce a conjugated hydrogel polymer mixture;
encapsulating the hydrophobic therapeutic agent with the cyclodextrin of the activated cyclodextrin product; and
precipitating particles from the conjugated hydrogel polymer mixture, the particles comprising a hydrogel polymer matrix having hydrophobic encapsulation sites therein.

18. The method of claim 17, wherein the step of activating a cyclodextrin feedstock comprises contacting the cyclodextrin with an imidazole.

19. The method of any claim 17 comprising crosslinking the hydrogel polymer matrix of the particles.

20. The method of claim 19, wherein the crosslinking comprises contacting the particles with glutaraldehyde.

21. The method of claim 17, wherein the step of precipitating particles from the conjugated hydrogel polymer mixture comprises contacting the conjugated hydrogel polymer mixture with acetone.

22. The method of claim 17, wherein the step of conjugating the activated cyclodextrin product comprises contacting the activated cyclodextrin product with the hydrogel polymer at a temperature of from 30 to 60° C. for a duration of 12 to 30 hours.

23. The method of claim 17, wherein the hydrogel polymer matrix comprises gelatin.

24. The method of claim 17, wherein the precipitating step comprises controlling precipitation conditions to create particles having a predetermined size distribution peak for diameter of from 50 nm to 1000 nm.
